(19) United States
(12) Patent Application Publication
(10) Pub. No.: US 2024/0335468 A1
(43) Pub. Date: Oct. 10, 2024

(54) TREATMENT OF RESPIRATORY CONDITIONS

(71) Applicant: HYPO-STREAM LIMITED, Melbourn, Cambridgeshire (GB)

(72) Inventors: Myles Dakin, Melbourn, Cambridgeshire (GB); Richard Aspinall, Melbourn, Cambridgeshire (GB); Thomas Kenny, Melbourn, Cambridgeshire (GB)

(21) Appl. No.: 18/578,811

(22) PCT Filed: Jul. 8, 2022
(86) PCT No.: PCT/EP2022/069154

§ 371 Date: Jan. 12, 2024

(30) Foreign Application Priority Data
Jul. 14, 2021 (GB) 2110146.4

(51) Int. Cl. A61K 33/20; A61K 9/00; A61K 9/08; A61K 47/02; A61P 1/18; A61P 11/00; A61P 19/02; A61P 31/14; A61P 37/02

(52) U.S. Cl. CPC A61K 33/20; A61K 9/0078; A61K 9/008; A61K 9/08; A61K 47/02; A61P 1/18; A61P 11/00; A61P 19/02; A61P 31/14; A61P 37/02

(57) ABSTRACT

The present invention relates to a hypochlorite solution for use in the prevention or treatment of a respiratory condition or disease in a patient; preferably a mammal, more preferably a human. The present invention also relates to a hypochlorite solution for use in the prevention or treatment of an inflammatory or auto-immune response, condition or disease in a patient, preferably a mammal, more preferably a human, wherein administration of the hypochlorite solution is by inhalation of the hypochlorite solution. The hypochlorite solution comprises hypochlorite in a concentration range of about 0.005-0.2 wt % (about 50-2000 ppm by wt).

### FIELD OF THE INVENTION

The present invention relates to a new approach to the prevention and treatment of respiratory conditions in a patient (e.g. mammals, especially humans) by use of hypochlorite solution. The present invention also relates to a new approach to the prevention and treatment of inflammation in a patient (e.g. mammals, especially humans) by inhalation of hypochlorite solution.

### BACKGROUND TO THE INVENTION

Respiratory conditions pose a significant challenge to human health worldwide. A recent WHO report (The Global Impact of Respiratory Disease—2017) has highlighted that:
An estimated 65 million people have moderate to severe chronic obstructive pulmonary disease (COPD), of which about 3 million die each year. This makes it the third leading cause of death worldwide. Furthermore, the number of patients with COPD is increasing.
About 334 million people suffer from asthma, the most common chronic disease of childhood, affecting 14% of children globally. The number of children with asthma is also growing.
Acute lower respiratory tract infections are among the top three causes of death and disability among children and adults. For example, respiratory tract infections caused by influenza kill between 250,000 and 500,000 people annually.
Tuberculosis (TB) is another respiratory disease that has historically been a challenge. In 2015, 10.4 million people developed TB. Of these, 1.4 million people have died from TB.

In particular, the unprecedented COVID-19 pandemic has added significant pressure to health establishments around the world.

There is therefore a pressing need to develop new treatments of respiratory conditions.

The use of aqueous chlorine in healthcare has been previously described in the context of the disinfection of surfaces and of contaminated wounds [see, for example, Bashford et al, Lancet 1917, 2: 595-597, Bunyan, Brit Med J. 1941, 4002-7; and Century Pharmaceuticals Inc., Dakin's solutions product information and material safety data sheet, 2011, http://www.dakins.net/index.html)].

However, any such use has been limited by the prevailing knowledge that toxicity is a problem associated with the use of aqueous chlorine in healthcare. This is especially true for respiratory tissue, which can be more sensitive compared to dermal tissue.

The present invention addresses this need by providing a new approach to the prevention and treatment of respiratory conditions in a patient (e.g. mammals, especially humans) by use of hypochlorite solution. The present invention also provides a new approach to the prevention and treatment of inflammation in a patient (e.g. mammals, especially humans) by inhalation of hypochlorite solution. It has been surprisingly found that a hypochlorite solution as described herein can be safely administered by inhalation and can be used to treat the conditions as described herein.

### SUMMARY OF THE INVENTION

According to an aspect of the present invention, there is provided a hypochlorite solution for use in the prevention or treatment of a respiratory condition or disease in a patient, preferably a mammal, more preferably a human. In particular, there is provided a hypochlorite solution for use in the prevention or treatment of a respiratory condition or disease in a patient, preferably a mammal, more preferably a human, wherein the hypochlorite solution comprises hypochlorite in a concentration range of about 0.005-0.2 wt % (about 50-2000 ppm by wt).

In an embodiment, the respiratory condition or disease may be acute respiratory distress syndrome (ARDS); asthma; bronchitis; chronic obstructive pulmonary disease (COPD); common cold; coronaviral diseases such as severe acute respiratory syndrome (SARS), COVID-19; cystic fibrosis; influenza; Middle East respiratory syndrome (MERS); pneumonia, such as viral pneumonia, bacterial pneumonia and ventilator-associated pneumonia; pulmonary fibrosis; rhinoviral diseases; sarcoidosis (e.g. affecting lungs); tuberculosis; or inflammation of lung tissue.

In an embodiment, the respiratory condition or disease may be an acute respiratory condition or disease.

In an embodiment, the administration may be by inhalation of the hypochlorite solution.

According to another aspect of the present invention, there is provided a hypochlorite solution for use in the prevention or treatment of an inflammatory or auto-immune response, condition or disease in a patient, preferably a mammal, more preferably a human, wherein administration of the hypochlorite solution is by inhalation of the hypochlorite solution. In particular, there is provided a hypochlorite solution for use in the prevention or treatment of an inflammatory or auto-immune response, condition or disease in a patient, preferably a mammal, more preferably a human, wherein the hypochlorite solution comprises hypochlorite in a concentration range of about 0.005-0.2 wt % (about 50-2000 ppm by wt).

In an embodiment, the inflammatory or auto-immune response, condition or disease may be arthritis such as osteoarthritis; pancreatitis; Sjogren's syndrome; or myasthenia gravis.

In an embodiment, the inflammatory or auto-immune response, condition or disease may be an acute inflammatory or auto-immune response, condition or disease.

In an embodiment, the administration of the hypochlorite solution by inhalation may be via a nebulizer or an inhaler.

In an embodiment, the hypochlorite may be sodium hypochlorite.

In an embodiment, the hypochlorite may be in a concentration range of about 0.005-0.2 wt % (about 50-2000 ppm by wt), more preferably about 0.01-0.1 wt % (about 100-1000 ppm by wt), even more preferably about 0.015-0.075 wt % (about 150-750 ppm), yet even more preferably about 0.025-0.075 wt % (about 250-750 ppm by wt), most preferably about 0.04-0.06 wt % (about 400-600 ppm by wt).

In an embodiment, the hypochlorite solution may further comprise sodium chloride.

In an embodiment, the sodium chloride may be in a concentration range of about 0.5-3.0 wt %, more preferably 0.5-1.5 wt %, even more preferably in a concentration range of about 0.6-1.3 wt %, yet even more preferably in a concentration range of about 0.7-1.2%, most preferably in a concentration range of about 0.8-1.0 wt %.

In an embodiment, the hypochlorite solution may have a pH of from about 5-11, preferably about 6-10, more preferably about 7-9, even more preferably about 7-8.

In an embodiment, the hypochlorite solution may be unbuffered.

In an embodiment, the hypochlorite solution may be buffered to a pH of from about 5-11, preferably about 6-10, more preferably about 7-9, even more preferably about 7-8.

In an embodiment, the buffer may be selected from the group consisting of a phosphate/phosphoric acid buffer, a borate/boric acid buffer and a citrate/citric acid buffer.

The hypochlorite solution may be an aqueous sodium hypochlorite solution comprising sodium hypochlorite, sodium chloride, and water to balance. Preferably the hypochlorite solution comprise 0.005-0.2 wt % sodium hypochlorite (about 50-2000 ppm by wt); 0.5-3.0 wt % sodium chloride; and water to balance. The concentrations of sodium hypochlorite and sodium chloride may be in accordance with those described above.

The hypochlorite solution may be an aqueous sodium hypochlorite solution consisting of 0.005-0.2 wt % sodium hypochlorite (about 50-2000 ppm by wt); 0.5-3.0 wt % sodium chloride; and water to balance. The concentrations of sodium hypochlorite and sodium chloride may be in accordance with those described above.

According to another aspect of the present invention, there is provided a method for preventing or treating a respiratory condition or disease comprising administering a therapeutically effective amount of hypochlorite solution as defined herein to a patient in need thereof.

According to another aspect of the present invention, there is provided a method for preventing or treating an inflammatory or auto-immune response, condition or disease comprising administering a therapeutically effective amount of hypochlorite solution as defined herein to a patient in need thereof.

According to another aspect of the present invention, there is provided a use of a hypochlorite solution as defined herein in the preparation of a medicament for preventing or treating a respiratory condition or disease in a patient.

According to another aspect of the present invention, there is provided a use of a hypochlorite solution as defined herein in the preparation of a medicament for preventing or treating an inflammatory or auto-immune response, condition or disease in a patient, wherein administration of the hypochlorite solution is by inhalation of the hypochlorite solution.

According to another aspect of the present invention, there is provided a kit comprising a hypochlorite solution as defined herein, and a nebulizer or an inhaler.

According to another aspect of the present invention, there is provided a pharmaceutical composition for use in the prevention or treatment of a respiratory condition or disease in a patient, preferably a mammal, more preferably a human, wherein the pharmaceutical composition comprises a hypochlorite solution as described herein, and wherein the pharmaceutical composition is administrable by inhalation. The pharmaceutical composition may comprise a propellant. The pharmaceutical composition may be a pharmaceutical aerosol composition suitable for inhalation.

According to another aspect of the present invention, there is provided a medicament container for use with an inhalation device, the medicament container containing a hypochlorite solution as described herein, and optionally a propellant. The inhalation device may be an inhaler. The inhalation device may be a nebuliser. The medicament container may be a pressurised medicament container, for example, a pressurised medicament container suitable for use with an inhaler.

The present invention provides new effective treatment options for respiratory, inflammatory and/or autoimmune conditions.

### DESCRIPTION OF THE FIGURES

The invention is further described in the following non-limiting figures:

FIG. 1 shows IL-6 cells in Example 1 dialysed against a control saline solution (0.85 wt %) to determine functionality of cytokine.

FIG. 2 shows IL-6 cells in Example 1 dialysed against a solution of Composition 1 to determine functionality of cytokine.

FIG. 3 shows IL-6 cells in Example 1 dialysed against a solution of Composition 2 to determine functionality of cytokine.

FIG. 4 shows IL-10 cells in Example 2 dialysed against a control saline solution (0.85 wt %) to determine functionality of cytokine.

FIG. 5 shows IL-10 cells in Example 2 dialysed against a solution of Composition 1 to determine functionality of cytokine.

FIG. 6 shows IL-10 cells in Example 2 dialysed against a solution of Composition 2 to determine functionality of cytokine.

FIG. 7 shows survival of MC9 cells following exposure of IL-10 to A (Composition 1) and B (Composition 2).

FIG. 8 shows survival of B9 cells following exposure of IL-6 to A (Composition 1) and B (Composition 2).

### DETAILED DESCRIPTION OF THE INVENTION

The following embodiments apply to all aspects of the present invention.

The present invention will now be further described. In the following passages, different aspects of the invention are defined in more detail. Each aspect so defined may be combined with any other aspect or aspects or embodiment or embodiments unless clearly indicated to the contrary. In particular, any feature indicated as being preferred or advantageous may be combined with any other feature or features indicated as being preferred or advantageous.

In the present application, a number of general terms and phrases are used, which should be interpreted as follows.

The term “treating”, as used herein, unless otherwise indicated, means reversing, attenuating, alleviating or inhibiting the progress of the disease or condition to which such term applies, or one or more symptoms of such disorder or condition. The term treating as used herein may also include prophylactic treatment, that is treatment designed to prevent the condition from occurring or minimize the likelihood of a condition occurring.

“Patient” includes humans, non-human mammals (e.g., dogs, cats, rabbits, cattle, horses, sheep, goats, swine, deer, and the like) and non-mammals (e.g., birds, and the like).

The term “acute”, as used herein, unless otherwise indicated, means a condition or disease lasting for a few days (e.g. about 1, 2, 3, 4, 5, 6 or 7 days) to a few weeks (e.g. about 1, 2, 3 or 4 weeks). An acute condition or disease is typically accompanied with an acute phase inflammatory response, or prevailing acute inflammatory cells (e.g. neutrophils) and acute inflammatory exudate.

The term “chronic”, as used herein, unless otherwise indicated, means a condition or disease lasting for a few months (e.g. about 1, 2, 3, 4, 5, 6, 7, 8, 9, 10, 11 or 12 months) to years (e.g. about 2, 3, 5, 10, 20, 30, 40, 50, 60, 70, 80, 90, 100 years). A chronic condition or disease is typically accompanied with a chronic inflammatory response with presence of macrophages, monocytes, lymphocytes, and with proliferation of blood vessels and connective tissue.

Acute conditions or diseases typically arise suddenly and last for a shorter duration compared to chronic conditions or diseases. For example, the acute condition or disease may be an acute phase response, an acute response mediated by the innate immune system or a systemic acute inflammatory response. The hypochlorite solution of the present invention is particularly suited for treating such acute conditions or diseases since the desired therapeutic effects of the hypochlorite solution have been found to take effect quickly (e.g. on the order of a few minutes to a few hours). The treatment can also be withdrawn quickly.

The hypochlorite solution of the present invention may be used in the prevention or treatment of a respiratory condition or disease. Preferably, the respiratory condition or disease is acute respiratory distress syndrome (ARDS); asthma; bronchitis; chronic obstructive pulmonary disease (COPD); common cold; coronaviral diseases such as severe acute respiratory syndrome (SARS), COVID-19; cystic fibrosis; influenza; Middle East respiratory syndrome (MERS); pneumonia, such as viral pneumonia, bacterial pneumonia and ventilator-associated pneumonia; pulmonary fibrosis; rhinoviral diseases; sarcoidosis (e.g. affecting lungs); tuberculosis; or inflammation of lung tissue.

COVID-19 includes the long term effects and symptoms of COVID-19, which is referred to herein as ‘Long COVID-19’. ‘Long COVID-19’ patients are patients who do not fully recover from COVID-19. ‘Long COVID-19’ patients continue to experience long-term effects or symptoms of the disease weeks, months, or even years (e.g. at least 1 month) after contracting COVID-19. ‘Long COVID-19’ patients experience these long-term effects and symptoms, despite an apparent absence of COVID-19 viral load.

The hypochlorite solution of the present invention may be used in the prevention or treatment of a respiratory condition or disease selected from chronic obstructive pulmonary disease (COPD); pneumonia, such as viral pneumonia, bacterial pneumonia and ventilator-associated pneumonia; and COVID-19.

Preferably, the respiratory condition or disease is an acute respiratory condition or disease.

Preferably, administration is by inhalation of the hypochlorite solution. For example, the inhalation may be via the nose and/or mouth.

The hypochlorite solution of the present invention may be used in the prevention or treatment of an inflammatory or auto-immune response, condition or disease in a patient, preferably a mammal, more preferably a human, wherein administration of the hypochlorite solution is by inhalation of the hypochlorite solution. For example, the inhalation may be via the nose and/or mouth. Preferably, the inflammatory or auto-immune response, condition or disease is arthritis such as osteoarthritis; pancreatitis; Sjogren's syndrome; or myasthenia gravis.

Preferably, the inflammatory or auto-immune response, condition or disease is an acute inflammatory or auto-immune response, condition or disease.

Without wishing to be bound by theory, it is thought that the hypochlorite solution of the present invention acts on the known initiators, mediators and regulators of inflammation in epithelial tissue, such as respiratory epithelium. The solution of the invention is thought to inhibit the release of inflammatory agents (e.g. cytokines and chemokines) from blood platelets, but does not prevent the platelets from aggregating. It is thought that the solution also attenuates the effect of cytokines, chemokines and other inflammatory mediators. Proof of concept that such an effect can be exploited in the treatment of a respiratory condition or disease, and/or an inflammatory or auto-immune response, condition or disease via inhalation, has been demonstrated in the Examples described herein. It has also been found that such an effect can be provided without serious safety issues, as demonstrated in the Examples described herein.

The inventors have surprisingly and unexpectedly discovered that the hypochlorite solution as described herein can selectively affect the function of particular anti-inflammatory cytokines. The data described herein demonstrates that the hypochlorite solution as described herein causes loss of IL-6 function in vitro. Conversely, the hypochlorite solution as described herein led to no significant change in IL-10 function in vitro.

As such, the hypochlorite solution of the present invention can disrupt normal cytokine activity in vitro thus providing a means of controlling cytokine-dependent cell signalling pathways. IL-6 is known to have pro-inflammatory activity. IL-10 is known to have anti-inflammatory activity. Without wishing to be bound by theory, these data indicate that use of the hypochlorite solution causes loss of IL-6 function whilst maintaining IL-10 function, which signals a net anti-inflammatory effect.

The inventors further demonstrate, for the first time, that inhalation of the hypochlorite solution as described herein provides significant relief for patients suffering from several respiratory conditions and diseases. For instance, the inventors have demonstrated that inhalation of the hypochlorite solution as described herein is an effective treatment for chronic obstructive pulmonary disease (COPD); pneumonia, COVID-19 and Long COVID-19. This is believed to be due to the net anti-inflammatory effect described above.

The hypochlorite solution for use in the prevention or treatment of a respiratory condition or disease and/or an inflammatory or auto-immune response, condition or disease is preferably administered by inhalation via a nebulizer. The type of nebulizer is not particularly limited provided that it is able to break up the hypochlorite solution into aerosol droplets for inhalation. The nebulizer may be connected to an outlet configured to deliver the hypochlorite solution to a patient via the nose and/or mouth. For example, the outlet may be a mouthpiece or a facemask.

Non-limiting examples of nebulizers include jet nebulizers (e.g. nebulizers connected to a supply of compressed air or oxygen, where flow of the compressed air or oxygen through the solution causes the generation of an aerosol), ultrasonic nebulizers (e.g. nebulizers connected to a piezoelectric vibrator, where the vibrations from the piezoelectric vibrator cause the generation of an aerosol) and mesh nebulizers (e.g. nebulizers having a membrane with fine holes, where forcing of the solution through the membrane causes the generation of an aerosol).

The hypochlorite solution for use in the prevention or treatment of a respiratory condition or disease and/or an inflammatory or auto-immune response, condition or disease is preferably administered by inhalation via an inhaler. For example, the inhaler may be a metered-dose inhaler. The metered-dose inhaler may be configured to deliver a set dose of the hypochlorite solution on each actuation of the metered-dose inhaler.

The hypochlorite solution for use in the prevention or treatment of a respiratory condition or disease and/or an inflammatory or auto-immune response, condition or disease may be provided in a medicament container that is suitable for use with an inhalation device. Medicament containers may include cartridges or canisters suitable for use with inhalation devices such as an inhaler or nebuliser.

The hypochlorite solution for use in the prevention or treatment of a respiratory condition or disease and/or an inflammatory or auto-immune response, condition or disease may be packaged into a cartridge containing a propellant, thus forming a cartridge comprising a hypochlorite solution and a propellant. Similarly, the hypochlorite solution for use in the prevention or treatment of a respiratory condition or disease and/or an inflammatory or auto-immune response, condition or disease may be packaged into a canister container a propellant, thus forming a canister comprising a hypochlorite solution and a propellant.

The propellant may be a chlorofluorocarbon or a hydrofluoroalkane, or the like.

The cartridge or canister may be used with an inhaler. The cartridge or canister may be detachable from the inhaler. The cartridge or canister may be attached to the inhaler prior to use.

The hypochlorite solution for use in the prevention or treatment of a respiratory condition or disease and/or an inflammatory or auto-immune response, condition or disease is preferably an aqueous solution.

The hypochlorite solution for use in the prevention or treatment of a respiratory condition or disease and/or an inflammatory or auto-immune response, condition or disease is preferably dilute sodium hypochlorite solution.

The hypochlorite solution may be a hypochlorite solution formulated to optimise therapeutic action and, simultaneously, avoid harmful actions. This can be achieved via dilution of a solution to provide a dilute hypochlorite solution.

Preferably, the hypochlorite solution contains hypochlorite in a concentration range of about 0.005-0.2 wt % (about 50-2000 ppm by wt), more preferably about 0.01-0.1 wt % (about 100-1000 ppm by wt), even more preferably about 0.015-0.075 wt % (about 150-750 ppm), yet even more preferably about 0.025-0.075 wt % (about 250-750 ppm by wt), most preferably about 0.04-0.06 wt % (about 400-600 ppm by wt). For example, the hypochlorite solution contains hypochlorite at about 0.05 wt % (about 500 ppm by wt).

The hypochlorite solution for use in the prevention or treatment of a respiratory condition or disease and/or an inflammatory or auto-immune response, condition or disease may further comprise sodium chloride, preferably in a concentration range of about 0.5-3.0 wt %, more preferably about 0.5-1.5 wt %, even more preferably in a concentration range of about 0.6-1.3 wt %, yet even more preferably in a concentration range of about 0.7-1.2%, most preferably in a concentration range of about 0.8-1.0 wt %.

The hypochlorite solution for use in the prevention or treatment of a respiratory condition or disease and/or an inflammatory or auto-immune response, condition or disease may have a pH of from about 5-11, preferably about 6-10, more preferably about 7-9, even more preferably about 7-8. In some embodiments, the pH may be about 5-6, about 6-7, about 8-9, about 9-10, or about 10-11. In embodiments, the pH of the hypochlorite solution is greater than 7.5. For example, in embodiments, the pH of the hypochlorite solution from 10-11. Alkaline pHs are generally preferred to ensure the presence of hypochlorite ion (ClO⁻). Acidification of hypochlorites generates hypochlorous acidm which is a different chemical entity.

Typically, when used, the pH of the hypochlorite solution is within a range encountered in normal physiology and disease processes. This is because the hypochlorite solution preferably auto-adjusts pH.

The hypochlorite solution for use in the prevention or treatment of a respiratory condition or disease and/or an inflammatory or auto-immune response, condition or disease may be unbuffered. In preferred embodiments, the hypochlorite solution for use in the prevention or treatment of a respiratory condition or disease and/or an inflammatory or auto-immune response, condition or disease is unbuffered. In other words, the hypochlorite solution is free of buffer agents. This allows the pH of the hypochlorite solution to be freely auto-adjusted at the site where it is administered. In embodiments of the invention, the composition is such that the pH of the hypochlorite solution to be freely auto-adjusted at the site where it is administered.

Alternatively, the hypochlorite solution for use in the prevention or treatment of a respiratory condition or disease and/or an inflammatory or auto-immune response, condition or disease may be buffered to a pH of from about 5-11, preferably about 6-10, more preferably about 7-9, even more preferably about 7-8. In some embodiments, the pH may be about 5-6, about 6-7, about 8-9, about 9-10, or about 10-11. The buffer may be any suitable buffer conventionally used in the pharmaceutical field, and is preferably selected from the group consisting of a phosphate/phosphoric acid buffer, a borate/boric acid buffer, and a citrate/citric acid buffer.

The hypochlorite solution for use in the prevention or treatment of a respiratory condition or disease and/or an inflammatory or auto-immune response, condition or disease is preferably free of stabilising agents.

The hypochlorite solution for use in the prevention or treatment of a respiratory condition or disease and/or an inflammatory or auto-immune response, condition or disease may be administered continuously. For example, such continuous administration may be applied to ventilated patients, patients in a hospital setting, sedated patients, or patients in a coma or induced coma. In some cases, the administration may be for a period of about 30 seconds to about 90 minutes. Preferably, the hypochlorite solution is administered for a period of about 1 minute to about 90 minutes, about 5 minutes to about 90 minutes, about 10 minutes to about 90 minutes, about 15 minutes to about 90 minutes, about 20 minutes to about 90 minutes, about 30 minutes to about 90 minutes, about 45 minutes to about 90 minutes, about 60 minutes to about 90 minutes, about 30 seconds to about 60 minutes, about 30 seconds to about 45 minutes, about 30 seconds to about 30 minutes, about 30 seconds to about 20 minutes, about 30 seconds to about 15 minutes, about 30 seconds to about 10 minutes, about 30 seconds to about 5 minutes or about 30 seconds to about 1 minute.

The hypochlorite solution for use in the prevention or treatment of a respiratory condition or disease and/or an inflammatory or auto-immune response, condition or disease may be administered once, twice, thrice or four times daily. Preferably, the hypochlorite solution is administered once daily.

The hypochlorite solution for use in the prevention or treatment of a respiratory condition or disease and/or an inflammatory or auto-immune response, condition or disease may be administered in a cycle comprising a treatment period of administration of the hypochlorite solution once, twice, thrice or four times daily for a period of a few days (e.g. about 1, 2, 3, 4, 5, 6 or 7 days) to a few weeks (e.g. about 1, 2, 3 or 4 weeks), followed by a period where the hypochlorite solution is not administered for a period of a few days (e.g. about 1, 2, 3, 4, 5, 6 or 7 days) to a few weeks (e.g. about 1, 2, 3 or 4 weeks). The cycle may be repeated at least twice. For example, the cycle may be repeated twice, thrice, four times, five times, six times, seven times, eight times, nine times, ten times, etc.

Beneficial effects for the prevention or treatment of a respiratory condition or disease and/or an inflammatory or auto-immune response, condition or disease are particularly noticeable where the patient is treated with a solution which has a concentration range of about 0.005-0.2 wt % (about 50-2000 ppm by wt), yet more preferably about 0.01-0.1 wt % (about 100-1000 ppm by wt), even more preferably about 0.025-0.075 wt % (about 250-750 ppm by wt) sodium hypochlorite; sodium chloride in a concentration range of about 0.5-1.5 wt %, preferably in a concentration range of about 0.6-1.3 wt %, more preferably in a concentration range of about 0.7-1.2%, most preferably in a concentration range of about 0.8-1.0 wt %; wherein the solution is unbuffered. Yet more preferably the solution consists of about 0.005-0.2 wt % (about 50-2000 ppm by wt), yet more preferably about 0.01-0.1 wt % (about 100-1000 ppm by wt), even more preferably about 0.025-0.075 wt % (about 250-750 ppm by wt) sodium hypochlorite; sodium chloride in a concentration range of about 0.5-1.5 wt %, preferably in a concentration range of about 0.6-1.3 wt %, more preferably in a concentration range of about 0.7-1.2%, most preferably in a concentration range of about 0.8-1.0 wt %; wherein the solution is unbuffered.

Beneficial effects for the prevention or treatment of a respiratory condition or disease and/or an inflammatory or auto-immune response, condition or disease are particularly noticeable where the patient is treated with a solution which has a concentration range of about 0.005-0.2 wt % (about 50-2000 ppm by wt), yet more preferably about 0.01-0.1 wt % (about 100-1000 ppm by wt), even more preferably about 0.025-0.075 wt % (about 250-750 ppm by wt) sodium hypochlorite; sodium chloride in a concentration range of about 0.5-1.5 wt %, preferably in a concentration range of about 0.6-1.3 wt %, more preferably in a concentration range of about 0.7-1.2%, most preferably in a concentration range of about 0.8-1.0 wt %; wherein the solution is buffered to a pH of from about 5-11, preferably about 6-10.

For example, a solution comprising 0.85% sodium chloride and 0.05% (500 ppm) sodium hypochlorite w/w has been found to be very beneficial in preventing and treating a respiratory condition or disease and/or an inflammatory or auto-immune response, condition or disease in a patient, preferably a mammal, more preferably a human. This range of concentrations has previously been considered in the medical literature to be toxic, and particularly so to respiratory tissue.

The hypochlorite should be very pure, e.g. ideally it should be generated electrolytically to ensure its purity as well as its safety and effectiveness.

The hypochlorite solution for use in the prevention or treatment of a respiratory condition or disease and/or an inflammatory or auto-immune response, condition or disease may be produced from a concentrated hypochlorite solution to be diluted before use in the respiratory condition or disease and/or an inflammatory or auto-immune response, condition or disease. Preferably, the concentrated hypochlorite solution to be diluted before use is a concentrated sodium hypochlorite solution. The concentrated hypochlorite solution when diluted to the appropriate dilution to give a hypochlorite solution may be used in the prevention or treatment of a respiratory condition or disease and/or an inflammatory or auto-immune response, condition or disease. These are as discussed and exemplified earlier.

The concentration of hypochlorite in the concentrated hypochlorite solution may be in the range of about 0.5 to 3 wt %. Furthermore, the concentrated hypochlorite solution may be buffered to a pH of from about 9-15, preferably about 11-13. Alternatively, the concentrated hypochlorite solution may be unbuffered.

The concentrated hypochlorite solution may be a stabilised sodium hypochlorite solution at 1% or 2% sodium hypochlorite, e.g. a disinfectant known as “Milton's Solution” comprising sodium chloride. The diluted hypochlorite solution may be a 2.5%-10% solution of Milton's solution diluted in water where the disinfectant solution is 2% sodium hypochlorite. The sodium chloride in the solution is typically at a concentration of 16.5%. Thus, the ratio by volume of the hypochlorite solution to water may be in the range of between 1 to 10 to 1 to 40. Alternatively, the dilute hypochlorite solution may be a 5% to 20% solution of Milton's solution diluted in water where the disinfectant solution is 1% sodium hypochlorite. In this case, the ratio by volume of the hypochlorite solution to water may be in the range of between 1 to 5 to 1 to 20.

In both cases, the predetermined amount of water and the predetermined amount of sodium hypochlorite solution may be such that the dilute disinfectant solution may be a stabilised sodium hypochlorite solution where the sodium hypochlorite is in a concentration range of about 0.005-0.2 wt % (about 50-2000 ppm by wt), more preferably about 0.01-0.1 wt % (about 100-1000 ppm by wt), even more preferably about 0.025-0.075 wt % (about 250-750 ppm by wt) sodium hypochlorite. The action of the sodium hypochlorite solution can provide stabilisation of the dilute disinfectant solution.

A device suitable for preparing a hypochlorite solution for use in the present invention is described in WO-A-2011/128862.

Various embodiments and optional features are described above. It will be appreciated that these embodiments and features can be combined in all viable permutations.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

### EXAMPLES

### Examples 1 and 2—In Vitro Studies on Cytokine Function

### Materials:

Solution of Composition 1—Hypochlorite at 0.005 wt %, in saline 0.85 wt %
Solution of Composition 2—Hypochlorite at 0.01 wt %, in saline at 0.8 wt %
IL-6 Dependent cell line B9 (Catalogue number 12121201, Mouse B cell hybridoma)
IL-10 Dependent cell line MC9 (ARCC® CRL-8306™, Mouse liver mast cell)
PrestoBlue® (cell-permeable fluorescent compound for use in cell-viability assays)

Method: Plasma samples were obtained from a volunteer and the cells were separated from the plasma. The plasma samples were placed in dialysis tubing whose pore size was less than the molecular weight of the cytokines. This was placed in 1000× excess (i.e. 1 ml in 1 L, 2 ml in 2 L etc.) of dialysis media.

The dialysis media was in either saline solution (0.85 wt %) as a control, Composition 1 or Composition 2 and was extracted after 5 minutes, 15 minutes, 30 minutes, 45 minutes and 60 minutes and then analysed. The cytokine functionality was thus determined.

Cytokine Function: Functional cell assays were carried out with patient plasma to investigate its ability to support the survival of cytokine dependent cell lines.

The serum was spiked with human recombinant cytokines. Heat-inactivated fetal bovine serum was used and was spiked with human recombinant IL-6 and IL-10 at 100 ng/ml.

The serum samples were dialysed with either 0.85 wt % saline solution, Composition 1 or Composition 2, as described above.

IL-6 function was assessed by determining the ability to maintain the growth of the IL-6 dependent cell line B9.

IL-10 Function was assessed by the ability to maintain the growth of the IL-10 dependent cell line MC9.

Cell viability was measured using fluorescence. A cell-permeable non-fluorescent compound was added to the assay. Viable cells maintain a reducing environment, which reduced the fluorescent compound, giving a colour change which fluoresces at 560 nm.

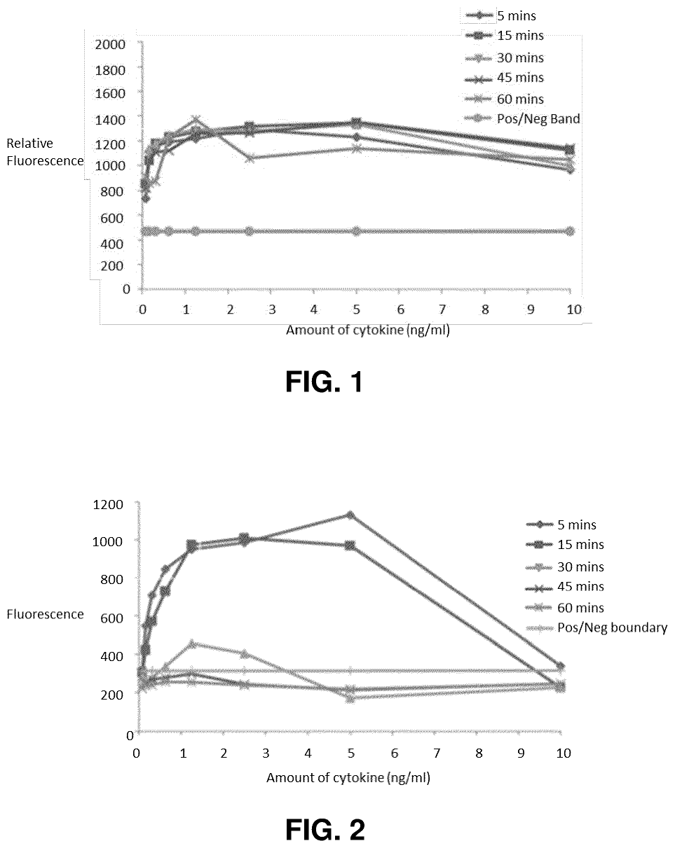

The degree of fluorescence was measured and directly correlates with the number of viable cells. The results are shown in FIGS. 1 to 8.

In some experiments, a high level of cytokine (i.e. 10 ng/ml) caused a decrease in cell viability. Without wishing to be bound by theory, the inventors postulate that this may be due to a prozone-like effect at high cytokine levels and/or that high levels of cytokine may cause cell death or the inhibition of cell metabolism in this system.

### Example 1—IL6

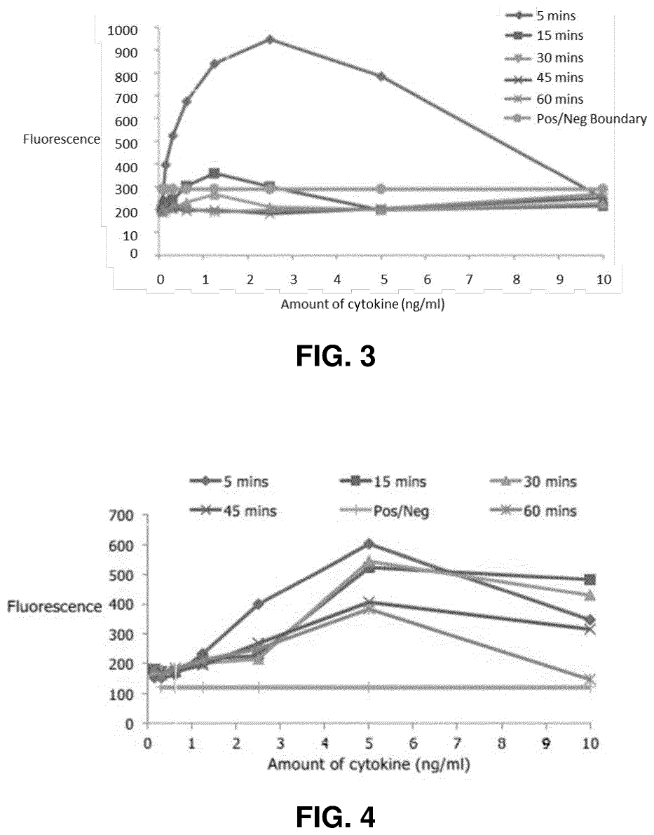

FIGS. 1 to 3 show the results from the cell-survival assay of the IL-6 spiked-serum samples dialysed against a control saline solution (FIG. 1), Composition 1 (FIG. 2) and Composition 2 (FIG. 3).

In these graphs, the line describing the Pos/Neg boundary indicates a value which is the mean of the negative control wells (no added cytokine, n=16) +3 standard deviations above this mean. Everything above this value is considered positive.

FIGS. 2 and 3 show that dialysis of the serum samples with Composition 1 or Composition 2 of the present invention and leads to a loss of function of the IL-6 molecule when measured in the cell survival assay.

### Example 2—IL10

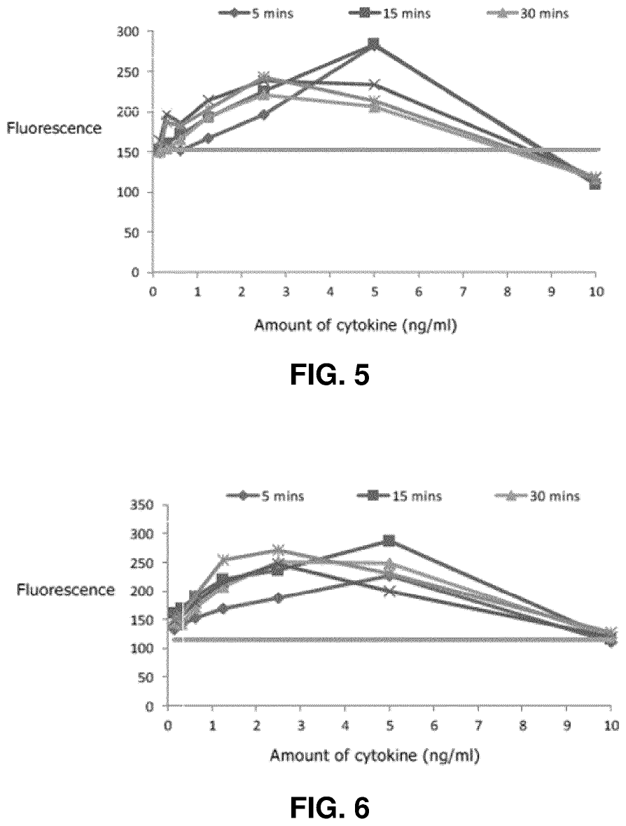

FIGS. 4 to 6 show the results from the cell survival assay of the IL-10 spiked-serum samples dialysed against a control saline solution (FIG. 4), Composition 1 (FIG. 5) and Composition 2 (FIG. 6).

In these graphs, the line describing the Pos/Neg boundary indicates a value which is the mean of the negative control wells (no added cytokine, n=16) +3 standard deviations above this mean. Everything above this value is considered positive.

The experimental data suggests that with dialysis of the serum samples with Compositions 1 or 2, IL-10 remains functional albeit with a slight loss of function of the IL-10 molecule when measured in the cell survival assay. IL-10 therefore remains functional as the loss of function is within expected range for an in vitro analysis with remaining net survival.

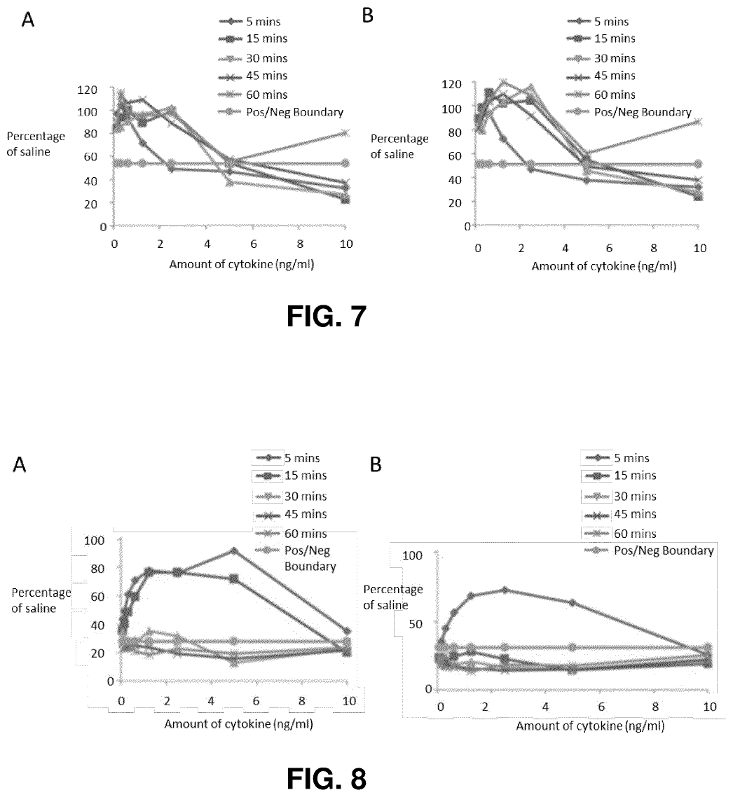

To evaluate this further, the data was analysed to look at the cell survival seen following exposure to Composition 1 or Composition 2 as a percentage of that achieved by the same quantity of cytokine following dialysis of 0.85 wt % saline solution (FIG. 7) and the results were compared with those obtained for IL-6 analysed in the same way (FIG. 8).

It can be seen from FIG. 7 that IL-10 receptor binding is retained ensuring the survival of MC9 cells. In contrast, in FIG. 8, there is almost a complete loss of function of IL-6 after 15 minutes exposure of Composition 1 or Composition 2. With complete loss of IL-6 function and negligible impact on IL-10 function, the combined effect is significantly anti-inflammatory. This comprises removal of a pro-inflammatory cytokine action and a relative increase in an anti-inflammatory cytokine action. The net effect is therefore anti-inflammatory.

### Examples 3 to 7—In Vivo Patient Efficacy Studies

Treatment comprised administration of aqueous sodium hypochlorite solution in saline (NaOCl: 0.05 wt % (500 ppm), NaCl: 0.85 wt %—referred to in Examples 3 to 7 as Composition 3), delivered by nebulizer.

### Example 3

Male. 5th decade. Pneumonia of viral and bacterial aetiology. Onset of pneumonia initially viral and then secondarily bacterial. Treated by 2×60 minute inhaled Composition 3 over four days. Diagnosis and resolution of consolidation in right and left lungs was confirmed by physician. Recovery was accelerated by continuing administration of nebulised Composition 3 over a period of four weeks. Return to work (clinical practice) was enabled. Impact of Composition 3 on viral and bacterial infection as well as inflammatory response in lungs. Rapid recovery hypothesised due to enhanced resolution of the innate inflammatory response and resolution of exudate.

### Example 4

Male of 4th decade. Treatment for pain/relief of pain and discomfort from chronic pancreatitis (inflammatory disease of viscera). Treatment with Composition 3 inhaled from nebuliser for 20 minutes each day. Resolution of symptoms over fourteen days and patient was able to cease prescription medication for retro-sternal and back pain. When Composition 3 pulmonary administration was halted, pain symptoms recurred and were eliminated by re-administration of nebulised Composition 3. Pathophysiology is unclear, but likely to be systemic manifestation of pulmonary administration of Composition 3.

### Example 5

Female in 9th decade. Treatment for prevention of acute episodes in chronic obstructive pulmonary disease. Observed increase/improvement in blood oxygen saturation. Patient was a long term COPD patient with increasingly frequent admissions to hospital with acute pulmonary/chest infections. Administration of nebulised Composition 3 commenced twice a day for 20 minutes. Frequency of acute episodes was reduced from weekly to every 6 weeks. Capillary blood oxygenation was seen to improve. Reduction of frequency of acute episodes and increased blood oxygenation saturation is consistent with reduced exudate, reduced microbial load and improved diffusion across alveolar membranes.

### Example 6

Female in 9th decade. Treatment for pain associate with inflammatory joint disease (osteoarthritis) and pain associated with an auto-immune disease (myasthenia gravis). Patient was taking diclofenac sodium (50 mg BD) as well as co-codamol (paracetamol codeine) PRN for morning pain on walking to bathroom to micturate. Pain was in lower back and hip joints, which have confirmed osteoarthritis as well as confirmed myasthenia gravis. Administration of Composition 3 inhaled via nebuliser for 20 minutes twice a day. After four days the patient was pain free in the mornings and not taking the diclofenac sodium 50 mg BD. This has been maintained. Hypothesised impact of Composition 3 on auto-immune disease and pain.

### Example 7

Female in 9th decade suffering with Sjogren's syndrome which was confirmed by a rheumatologist. Principal complaint was dry mouth and severe pain in mouth and pharynx. Pain has been severe enough to make eating any foods too painful resulting in weight loss.

Sjogren's disease is an autoimmune disease whereby the pathologic condition results in antibodies that target the body's own moisture producing glands. These include salivary, lachrymal with other effects on the lungs, kidneys and nervous system. Muscle tiredness, impact on thyroid function, numbness of arms and legs is also reported.

Administration of Composition 3 was via a nebuliser to produce a vapour of the formula as well as a mouthrinse. Administration was every 6 hours for 20 to 30 minutes.

Painful and dry mouth symptoms have resolved to a degree that allows pain-free and non-dry mouth eating of all food types and consistencies including spiced recipes usually associated with acute pain and prolongation of worsening symptoms. Other general symptoms have improved and movement and sitting is more comfortable.

During the study, the treatment was withdrawn with a recurrence of painful symptoms and feeling of illness. The medication was re-introduced with successful resolution of symptoms within five days.

Prior to treatment, the patient had unsuccessfully tried corticosteroids prescribed by a specialist in oral medicine.

### Example 8—In Vivo Patient Safety Studies

Treatment comprised administration of aqueous sodium hypochlorite solution in saline (NaOCl: 0.05 wt % (500 ppm), NaCl: 0.85 wt %—referred to in Example 8 as Composition 3), delivered by nebulizer.

### Overall Study Plan

This study was designed as a Phase 1, open-label, exposure-escalation study in approximately 18 healthy volunteers who were 2 to 80 years old. Three exposure cohorts were planned.

Cohort one consisted of 2 adults:

Initially a single healthy volunteer. Inhalation of Composition 3 via nebuliser. Monitoring of Oxygen saturation, pulse rate, blood pressure and performance of coordination tests. Inhalation programme 30 seconds followed by 30-minute observation. 1 minute followed by 30-minute observation. The observation periods were kept at 30-minutes and the period of administration was increased to 5 minutes, 10 minutes, 15 minutes, 20 minutes, 30 minutes. On a subsequent day the administration period was increased to 45 minutes and 90 minutes.

This programme was repeated with a further healthy volunteer after a seven-day observation period on the first volunteer.

Both volunteers were followed up for 6 months, with no further inhalations and monitored for adverse reactions and any longer-term effects.

Cohort two consisted of cohort one plus an additional 4 adults:

The programme was then extended to a further group of healthy volunteers and following the induction phase of administrations for 30 secs, 1 minute, 5 minutes, 10 minutes and then 15 minutes, a daily administration time of 20 minutes was recommended based upon the assumption of the II-6 neutralisation time of 15 minutes with the Composition 3 seen in the mechanistic study plus a circulation time of 4-5 minutes for blood circulation around the body.

After 1 week of observed administration. The healthy volunteers self-administered during a three-month observation period and followed up over a period of 2 years and monitored for adverse reactions and any longer-term effects.

Cohort three consisted of 12 adults and children:

The programme was then extended to a further group of healthy volunteers at the extremes of age and following the induction phase of administrations for 5 minutes, 10 minutes and then 15 minutes, a daily administration time of 20 minutes. The healthy volunteers self-administered during a three-month observation period and followed up over a period of three months.

### Selection of Population

### Inclusion Criteria:

Provided written, signed informed consent, or, in the case of participants under the age of 18, written informed consent by a parent or legal guardian, after the nature of the study had been explained, and prior to any research-related procedures.
Agreed to comply with all study procedures.
Were in generally good health as evidenced by physical examination.

### Exclusion Criteria:

Donated blood or plasma within 30 days prior to the study drug administration.
Used any over-the-counter (OTC) medication, including vitamins, within 7 days prior to the administration of study drug, without evaluation and approval by the investigator.
Used any prescription medication within 14 days prior to the administration of study drug without evaluation and approval by the investigator.
Any uncontrolled pulmonary condition.

### Treatments

Subjects (or their legally authorized representative) could have withdrawn their consent to participate in the study at any time without prejudice. The investigator was to withdraw from the study any subject who requested to be withdrawn. A subject's participation in the study was to be discontinued at any time at the discretion of the investigator and in accordance with his/her clinical judgment.

If a subject failed to return for scheduled visits, a documented effort was to be made to determine the reason. If the subject could not be reached by telephone within 7 days, a certified letter was to be sent to the subject (or the subject's legally authorized representative, if appropriate) requesting contact with the investigator. This information was to be recorded in the study records.

### Cohort One:

Inhalation of Composition 3 via nebuliser.
Inhalation programme 30 seconds followed by 30-minute observation.
1 minute followed by 30-minute observation.
The observation periods were kept at 30-minutes and the period of administration was increased to 5 minutes, 10 minutes, 15 minutes, 20 minutes, 30 minutes.
On a subsequent day the administration period was increased to 45 minutes and 90 minutes.
After a further seven-day observation period the same schedule was repeated on the second volunteer.

### Cohort Two:

Induction phase of administrations for 30 secs, 1 minute, 5 minutes, 10 minutes and then 15 minutes, each followed by a 30 minute observation period.
Then from day 2, a daily administration time of 20 minutes.

### Cohort Three:

Induction phase of administrations for 5 minutes, 10 minutes and then 15 minutes, each followed by a 30 min observation phase.
Then from day 2, a daily administration time of 20 minutes.

Composition 3 was supplied as a sterile aqueous solution. Composition 3 was nebulised at a concentration of 500 ppm.

This was to be an open-label study; no randomization was to be used in assigning subjects to each cohort.

All prescription and OTC medications taken by a subject for 30 days before Screening were to be recorded on the designated CRF. The investigator could have prescribed additional medications during the study as long as the prescribed medication was not prohibited by the protocol. In the event of an emergency, any needed medications could have been prescribed without prior approval but the Sponsor's medical monitor must have been notified of the use of any contraindicated medications immediately thereafter. Any concomitant medications added or discontinued during the study were to be recorded on the CRF.

Use of any other investigational product or investigational medical device was to be prohibited within 30 days before Screening and until all scheduled study assessments were completed.

OTC medications and vitamins were to be prohibited within 7 days before Day 1 (study administration) and until all scheduled study assessments were completed without evaluation and approval by the investigator.

Use of any prescription medication was to be prohibited within 14 days before Day 1 and until all scheduled study assessments were completed without evaluation and approval by the investigator.

All used and unused drug containers were to be kept by the investigator. The quantity dispensed, returned, used, lost, etc., was to be recorded on the dispensing log provided for the study.

The PI was to be responsible for maintaining accurate records (including dates and quantities) of study drug received, subjects to whom study drug was dispensed (subject-by-subject dose specific accounting), and study drug lost or accidentally or deliberately destroyed. The investigator was to retain all unused or expired study supplies until the study monitor confirmed the accountability data.

Unused study drug could have been destroyed on site, per the site's standard operating procedures, but only after the Sponsor granted approval for drug destruction. The monitor was to account for all study drug in a formal reconciliation process prior to study drug destruction. All study drug destroyed on site was to be documented. Documentation was to be provided to the Sponsor and was to be retained in the investigator's study files. If a site was unable to destroy study drug appropriately, the site could have returned unused study drug to the Sponsor upon request. The return of study drug or study drug materials was to be accounted for by the Sponsor.

### Safety and Tolerability Variables

Safety was to be assessed by examining the incidence of all treatment-emergent adverse events (hereafter referred to as AEs) reported during the study period and clinically significant changes in vital signs.

Vital signs were to be measured after resting for 5 minutes and included seated systolic blood pressure (SBP) and diastolic blood pressure (DBP) measured in mmHg, heart rate in beats per minute, respiration rate in breaths per minute and oxygen saturation.

According to the ICH definition, an adverse event (or adverse experience) is “any untoward medical occurrence in a patient or clinical investigation subject administered a pharmaceutical product, and that does not necessarily have a causal relationship with this treatment. An AE can therefore be any unfavourable and unintended sign (including an abnormal laboratory finding), symptom, or disease temporally associated with the use of a medicinal (investigational) product, whether or not considered related to the IP.”

An adverse drug reaction (ADR) is described by the ICH as “all noxious and unintended responses to a medicinal product related to any dose.” This means that a causal relationship between a medicinal product and an AE is at least a reasonable possibility, i.e., the relationship cannot be ruled out.

An AE could have included intercurrent illnesses or injuries that represent an exacerbation (increase in frequency, severity, or specificity) of pre-existing conditions (e.g., worsening of asthma). Whenever possible, it was preferable to record a diagnosis as the AE term rather than a series of terms relating to a diagnosis.

The reporting period for nonserious AEs was to be the period from the first administration of study drug through Week 26 or early termination. If a nonserious AE remained unresolved at the conclusion of the study, the PI and Sponsor's medical monitor were to make a joint clinical assessment as to whether continued follow-up of the AE was warranted and the results of this assessment were to be documented. Resolution was to be defined as the return to baseline (Screening) status or stabilization of the condition with the expectation that it remained chronic.

The investigator was to assess AEs for severity, for relationship to study drug, and as to whether the event met one or more of the definitions of an SAE.

The investigator was to determine the severity of each AE and was to record it on the source documents and AE CRF using the categories defined below.

### Statistical Plan

Due to the exploratory nature of this study and small sample size overall, no formal statistical tests were to be performed for inference. Inferential statistics were to be used for descriptive purposes only. Unless specified otherwise, all summaries were to be comprised of standard descriptive statistics. Standard descriptive statistics were to include the following:
For continuous parameters: number of observations (or subjects), mean, standard deviation, median, minimum, and maximum.
For categorical parameters: frequency and percent of observations in each category.

Both efficacy and safety data were to be summarized by individual cohort and all cohorts combined.

Determination of Sample Size: A total of 18 subjects, treated in 3 cohorts, were planned for this study. A total cohort size of 18 subjects equally balanced across genders and with a wide age range, was deemed sufficient to meet the objectives of this study.

No formal sample size calculations or power determinations were to be conducted.

Planned Analysis: All subjects who received any amount of study drug and had post dose safety data were to be included in the safety analyses.

Handling of Drop out and missing data: All analyses were to be presented based on observed data only.

Data Monitoring Committee: A DMC was to be used in this study to monitor the safety of subjects in this first clinical study of Composition 3. Planned reviews were to occur after the completion of the induction phase of each cohort and at the end of each cohort's dosing and follow-up periods.

Safety Analyses: Safety was to be assessed by examining the incidence of all AEs reported during the study period and clinically significant changes in vital signs.

Only AEs reported during the study period were to be included in AE summaries. An AE was to be defined as any AE of new occurrence, increased in frequency, or worsened in severity following study drug administration. If the onset of an AE was missing and the AE resolution was either after the dose date or missing, then the AE was to be considered treatment emergent. Any AEs judged by the investigator as possibly or probably related to study drug were to be considered drug related. If relationship to study drug was missing, the AE was to be considered drug related.

The incidence and severity of AEs for all AE and all drug-related AEs was to be summarized by system organ class (SOC), preferred term, and severity. For those AEs that occurred more than once during the study, the maximum severity was to be used to summarize the AEs by severity. Subjects who reported multiple events that coded to a common preferred term or SOC were to be counted only once per preferred term or SOC.

AEs that were assessed by the investigator as possibly or probably related to study drug were to be summarized by SOC, preferred term, and maximum severity. Drug-related AEs were to be summarized similarly to AEs.

### Changes in Conduct of the Study or Panned Analysis

Of the 18 subjects enrolled in the study all met the eligibility criteria as defined by the protocol and received treatment as appropriate to the escalation schedule appropriate to their cohort.

### Study Subjects

| | Cohort | Cohort | Cohort | Com- |
|---|---|---|---|---|
| Characteristic | 1 | 2 | 3 | bined |
| n | 2 | 4 | 12 | 18 |
| Gender | | | | |
| Male | 2 (100%) | 3 (75%) | 4 (33%) | 9 (50%) |
| Female | 0 (0%) | 1 (25%) | 8 (66%) | 9 (50%) |
| Age at baseline | | | | |
| Mean (SD) | 53 (4.2) | 47.75 | 43.9 | 45.7 |
| | | (7.3) | (24.7) | (20.6) |
| Median | 53 | 47.5 | 41 | 42 |
| Min, Max | 50, 56 | 41, 55 | 3, 80 | 3, 80 |

### Safety Evaluations

Extent of Exposure: All subjects were exposure to Composition 3 according to their cohort.

Adverse Events: 4 subjects reported adverse events during the initial parts of induction phase of treatment (cohorts one and two, mild cough or feeling of wanting to cough), not experienced on subsequent exposure. Patients in cohort three were warned of this possibility and none reported it.

1 subject with underlying asthma report a sensation similar to asthma symptoms, no wheeze, no decrease in PEFR, treatment was paused for 1 week and re-introduced with no recurrence of sensation.

All subjects experienced increased expectoration after dosing.

No other adverse events occurred during the 1 hour period immediately following treatment.

No adverse events occurred during the follow up periods that were thought to be related to treatment.

Listing of Serious Adverse Events: There were no SAEs during the duration of the treatment or follow phases of this study.

| Characteristic | N |
|---|---|
| Mild cough/sensation of wanting to cough | 4 |
| Increase in expectoration | 18 |
| Asthma like sensation | 1 |

### Summary of Safety Studies

Summary of Results for the Phase 1, Open-label, Exposure-Escalation study to Evaluate the Safety and Tolerability of Nebulised Composition 3 in healthy volunteers:
i. Sample size n=18
ii. Serious Adverse events—0
iii. Adverse events during induction—4 (all grade 1 events)
iv. Adverse events during or in the immediate 1 h period following treatment—18 (all grade 1 event)
v. Other Adverse events thought to be related to treatment—0
vi. Age range 3-80 yrs
vii. Longest period of continuous administration—90 mins
viii. Mode period of continuous administration—20 minutes
ix. Maximum duration of repeated daily 20 minutes administration—over 4 years

Overall, the present invention has demonstrated that hypochlorite compositions according to the present invention are safe and well tolerated when administered via the inhalation route. The present invention has also demonstrated that in vitro data demonstrating anti-inflammatory properties leads to in vivo clinical improvement in patients with respiratory, inflammatory and/or autoimmune conditions. The present invention provides new effective treatment options for respiratory, inflammatory and/or autoimmune conditions.

### Example 9—In Vivo Patient Treatment of COVID-19 Study

Treatment comprised administration of aqueous sodium hypochlorite solution in saline (NaOCl: 0.05 wt % (500 ppm), NaCl: 0.85 wt %—referred to in Example 9 as Composition 3), delivered by nebulizer.

Each patient enrolled tested positive for COVID-19 before participating in the study, and displayed symptoms of COVID-19. Composition 3 was administered to each patient by nebuliser for the time periods indicated in the table below. Frequency of inhalation is indicated as (number of times per day x duration of each treatment session). Where administration was 4 times daily, Composition 3 was administered every 6 hours. Where administration was 3 times daily, Composition 3 was administered every 8 hours.

The treatment was administered at the indicated frequency on consecutive days until a negative lateral flow test result was obtained.

| | | | | | # | | # | |
|---|---|---|---|---|---|---|---|---|
| | | | | | Days | Symptom | Days to | |
| | | | # Vaccine | Frequency of | to −ve | Free After | no | |
| Patient | Age | Gender | Doses | inhalation | LFT | Treatment? | symptoms | Comments |
| 1 | 56 | M | 3 | 4 × 30 mins | 4 | Y | 2 | Rapid recovery |
| 2 | 58 | M | 3 | 2 × 30 mins | 6 | Y | 4 | Rapid recovery |
| 3 | 46 | F | 3 | 4 × 30 mins | 1 | Y | 4 | Rapid recovery |
| 4 | 67 | M | 3 | 3 × 30 mins | 4 | Y | 2 | Rapid recovery |
| 5 | 67 | F | 3 | 3 × 30 mins | 4 | Y | 2 | Rapid recovery |
| 6 | 9 | F | 2 | 3 × 20 mins | 3 | Y | 3 | Rapid recovery |
| 7 | 52 | F | 2 | 3 × 20 mins | 4 | Y | 4 | Rapid recovery |
| 8 | 55 | M | 2 | 3 × 20 mins | 4 | Y | 4 | Rapid recovery |
| 9 | 44 | F | 2 | 3 × 30 mins | 4 | Y | 3 | Rapid recovery |
| 10 | 15 | M | 2 | 3 × 30 mins | 2 | Y | 2 | Elite athlete |
| 11 | 34 | M | 3 | 3 × 30 mins | 4 | Y | 2 | Rapid recovery |
| 12 | 77 | F | 3 | 4 × 20 mins | 7 | Y | 2 | Rapid recovery; |
| | | | | | | | | Second infection |
| 13 | 84 | M | 3 | 4 × 20 mins | 10 | Y | 2 | Rapid recovery; |
| | | | | | | | | Second infection |

#: Number;
Vaccine Doses: Doses of a UK approved vaccine received by the patient prior to enrolment in study;
−ve LFT: negative lateral flow test

Each enrolled patient become symptom free within 2-4 days from the date Composition 3 was first administered (i.e. from the date dose 1 of Composition 3 was administered). Each patient was symptom free before receiving a negative lateral flow test (LFT). These data are indicative that Composition 3 administered via inhalation is an effective treatment for the symptoms of COVID-19, and reduces recovery time.

### Example 10—In Vivo Patient Treatment of ‘Long’ COVID-19 Study

Treatment comprised administration of aqueous sodium hypochlorite solution in saline (NaOCl: 0.05 wt % (500 ppm), NaCl: 0.85 wt %—referred to in Example 10 as Composition 3), delivered by nebulizer.

Each patient enrolled had previously tested positive for COVID-19, and was suffering from symptoms of ‘Long COVID-19’ for at least 1-month from the date of a positive COVID-19 test. Composition 3 was administered to each patient by nebuliser for the time periods indicated in the table below. Frequency of inhalation is indicated as (number of times per day x duration of each treatment session). Where administration was 4 times daily, Composition 3 was administered every 6 hours. Where administration was 3 times daily, Composition 3 was administered every 8 hours.

The treatment was administered at the indicated frequency on consecutive days until the patient reported no symptoms.

| | | | | Long Covid | Frequency | Symptom | #Days to | |
|---|---|---|---|---|---|---|---|---|
| | | | #Vaccine | Duration | of | Free After | no | |
| Patient | Age | Gender | Doses | (#Months) | inhalation | Treatment? | symptoms | Comments |
| 1 | 19 | F | 3 | 1.5 | 3 × 30 mins | Y | 4 | Full recovery; |
| | | | | | | | | Continued |
| | | | | | | | | treatment |
| 2 | 41 | M | 3 | 6 | 3 × 30 mins | Y | 7 | Full recovery; |
| | | | | | | | | Back to gym |
| 3 | 54 | M | 3 | 6 | 4 × 30 mins | Y | 4 | 90% recovery; |
| | | | | | | | | Asthma |
| | | | | | | | | improved |
| 4 | 74 | M | 3 | 1.5 | 2 × 30 mins | Y | 2 | Rapid |
| | | | | | | | | recovery |

#Number;
Vaccine Doses: Doses of a UK approved vaccine received by the patient prior to enrolment in study;

Long COVID-19 symptoms were alleviated in the patients, with the exception of two patients who made partial recoveries. Two patients made a full recovery from long COVID-19, and one patient made a near full (90%) recovery with mild asthma symptoms remaining. These data indicate that Composition 3 administered via inhalation is an effective treatment for symptoms of Long COVID-19 and improves the level of recovery achieved.

1. A method for preventing or treating a respiratory condition or disease, the method comprising administering a therapeutically effective amount of hypochlorite solution to a patient, preferably a mammal, more preferably a human, wherein the hypochlorite solution comprises hypochlorite in a concentration range of about 0.005-0.2 wt % (about 50-2000 ppm by wt).

2. A method according to claim 1, wherein the respiratory condition or disease is acute respiratory distress syndrome (ARDS); asthma; bronchitis; chronic obstructive pulmonary disease (COPD); common cold; coronaviral diseases such as severe acute respiratory syndrome (SARS), COVID-19; cystic fibrosis; influenza; Middle East respiratory syndrome (MERS); pneumonia, such as viral pneumonia, bacterial pneumonia and ventilator-associated pneumonia; pulmonary fibrosis; rhinoviral diseases; sarcoidosis (e.g. affecting lungs); tuberculosis; or inflammation of lung tissue.

3. A method according to claim 1, wherein the respiratory condition or disease is an acute respiratory condition or disease.

4. A method according to claim 1, wherein administration is by inhalation of the hypochlorite solution.

5. A method for preventing or treating an inflammatory or auto-immune response, condition or disease, the method comprising administering a therapeutically effective amount of hypochlorite solution to a patient in need thereof, preferably a mammal, more preferably a human, wherein the hypochlorite solution comprises hypochlorite in a concentration range of about 0.005-0.2 wt % (about 50-2000 ppm by wt) and wherein administration of the hypochlorite solution is by inhalation of the hypochlorite solution.

6. A method according to claim 5, wherein the inflammatory or auto-immune response, condition or disease is arthritis such as osteoarthritis; pancreatitis; Sjogren's syndrome; or myasthenia gravis.

7. A method according to claim 5, wherein the inflammatory or auto-immune response, condition or disease is an acute inflammatory or auto-immune response, condition or disease.

8. A method according to claim 4 or claim 5, wherein the administration of the hypochlorite solution by inhalation is via a nebulizer or an inhaler.

9. A method according to claim 1 or claim 5, wherein the hypochlorite is sodium hypochlorite.

10. A method according to claim 1 or claim 5, wherein the hypochlorite is in a concentration range of about 0.01-0.1 wt % (about 100-1000 ppm by wt), more preferably about 0.015-0.075 wt % (about 150-750 ppm), even more preferably about 0.025-0.075 wt % (about 250-750 ppm by wt), most preferably about 0.04-0.06 wt % (about 400-600 ppm by wt).

11. A method according to claim 1 or claim 5, wherein the hypochlorite solution further comprises sodium chloride.

12. A method according to claim 11, wherein the sodium chloride is in a concentration range of about 0.5-3.0 wt %, more preferably 0.5-1.5 wt %, even more preferably in a concentration range of about 0.6-1.3 wt %, yet even more preferably in a concentration range of about 0.7-1.2%, most preferably in a concentration range of about 0.8-1.0 wt %.

13. A method according to claim 1 or claim 5, wherein the hypochlorite solution is an aqueous sodium hypochlorite solution comprising (e.g. consisting of) sodium hypochlorite, sodium chloride, and water to balance.

14. A method according to claim 1 or claim 5, wherein the hypochlorite solution has a pH of from about 5-11, preferably about 6-10, more preferably about 7-9, even more preferably about 7-8.

15. A method according to claim 1 or claim 5, wherein the hypochlorite solution is unbuffered.

16. A method according to claim 1 or claim 5, wherein the hypochlorite solution is buffered to a pH of from about 5-11, preferably about 6-10, more preferably about 7-9, even more preferably about 7-8.

17. A method according to claim 16, wherein the buffer is selected from the group consisting of a phosphate/phosphoric acid buffer, a borate/boric acid buffer and a citrate/citric acid buffer.

18-21. (canceled)

22. A kit comprising a hypochlorite solution as defined in claim 1 or claim 5, and a nebulizer or an inhaler.

23. A medicament container for use with an inhalation device, the medicament container containing a hypochlorite solution as defined in claim 1 or claim 5, and optionally a propellant.

24. (canceled)
